(19) United States
(12) Patent Application Publication
(10) Pub. No.: US 2012/0207856 A1
(43) Pub. Date: Aug. 16, 2012

(54) MSH3 Expression Status Determines the Responsiveness of Cancer Cells to the Chemotherapeutic Treatment with PARP Inhibitors and Platinum Drugs

(75) Inventors: Ajay Goel, Dallas, TX (US); C. Richard Boland, Dallas, TX (US); Minoru Koi, Dallas, TX (US); Masanobu Takahashi, Richardson, TX (US)

(73) Assignee: Baylor Research Institute, Dallas, TX (US)

(21) Appl. No.: 13/370,752

(22) Filed: Feb. 10, 2012

Related U.S. Application Data
(60) Provisional application No. 61/442,192, filed on Feb. 12, 2011.

(51) Int. Cl. A61K 33/24; A61K 31/255; A61K 31/196; A61K 31/675; A61K 31/655; C07K 14/47; A61K 31/4745; C12Q 1/68; G01N 33/53; G01N 33/566; G01N 33/577; A61P 35/00; A61K 31/175; A61K 31/704

(52) U.S. Cl. 424/649; 514/589; 514/517; 514/567; 514/110; 514/151; 514/34; 514/283; 435/6.12; 436/501; 435/7.23; 530/350

(57) ABSTRACT

Methods for treating a patient at risk for or diagnosed with colorectal cancer are disclosed herein. The method of the present invention determines the overall expression of MSH3 in cells suspected of being colorectal cancer cells from the patient and predicting the efficacy of therapy with a genotoxic anti-neoplastic agent for treating the patient, wherein a decrease in the overall expression of MSH3 in the patient cells when compared to the expression of MSH3 in normal colorectal cells indicates a predisposition to responsiveness to genotoxic anti-neoplastic agent therapy, wherein the therapy comprises administering an effective amount of the genotoxic anti-neoplastic agent therapy to patients.

### CROSS-REFERENCE TO RELATED APPLICATIONS

This application claims the benefit of U.S. provisional patent application No. 61/442,192, filed Feb. 12, 2011 the contents of which are incorporated by reference herein.

### STATEMENT OF FEDERALLY FUNDED RESEARCH

This invention was made with U.S. Government support under Contract Nos. R01 CA72851 and CA129286 awarded by the National Cancer Institute (NCI)/National Institutes of Health (NIH). The government has certain rights in this invention.

### TECHNICAL FIELD OF THE INVENTION

The present invention relates in general to the field of cancer detection, prognosis and treatment, and more particularly, to methods for detecting the susceptibility of colorectal cancer cells to DNA damaging agents.

### INCORPORATION-BY-REFERENCE OF MATERIALS FILED ON COMPACT DISC

None.

### REFERENCE TO A SEQUENCE LISTING

None.

### BACKGROUND OF THE INVENTION

Without limiting the scope of the invention, its background is described in connection with biomarkers for colon and gastroenterological cancer detection.

U.S. Pat. No. 7,252,955 issued to Pant et al. (2007) discloses an immunological assay and kit for colon cancer screening. Fecal glycoproteins are extracted from individual samples such that immunogenicity is maintained. The purified fecal glycoproteins are reacted with antibodies to Colon and Ovarian Tumor Antigen (COTA). The mucin antigen COTA is specifically present in colorectal cancer tissue and not in normal colons. The amount of COTA in the fecal sample is determined and used to indicate the presence of colon cancer.

U.S. Pat. No. 7,575,928 issued to Lin et al., discloses genes for diagnosing colorectal cancer. Briefly, this patent provides genes for diagnosing colorectal cancer, by searching for gene sequences by: (1) deriving epithelium cells from normal intestines, polypus of intestines and colorectal cancer tissue; (2) collecting genes with highly differential gene expression by Suppression Subtractive Hybridization (SSH), and building library; (3) deriving colonies with relatively high signal intensities from cancer tissue; (4) collecting more clinically cancer tissues by Northern Hybridization, real-time Polymerase Chain Reaction (PCR) combined with analysis of bioinformation to affirm variation between differential gene expression; and (5) selecting the most suitable genes from said library, and using the gene sequence as reagent provides the effects of early diagnosis, specificity, highly sensitivity and safety.

U.S. Pat. No. 7,022,472, issued to Robbins et al., discloses mutations in human MLH1 and human MSH2 genes useful in diagnosing colorectal cancer. Briefly, it was found that variants of the human MLH1 and MSH2 genes could be used to diagnose hereditary non-polyposis colorectal cancer (HNPCC) and/or determine a patient's susceptibility to developing HNPCC are also provided. Methods and compositions for identifying new variant MLH1 of MSH2 genes are also provided. In addition, experimental models for hereditary non-polyposis colorectal cancer comprising these variant genes were provided.

Finally, U.S. Patent Publication No. 20090305277 filed by Baker et al., describes a method of predicting a likelihood that a human patient diagnosed with cancer based on determining an expression level of at least one gene selected from the group consisting of AURKB, Axin 2, B1K, BRAF, BRCA2, BUB1, C20 orf1, C200RF126, CASP9, CCNE2 variant 1, CDC2, CDC4, CENPA, CENPF, CLIC1, CYR61, Cdx2, Chk1, DLC1, DUSP1, E2F1, EGR3, E124, ESPL1, FBXO5, FGF2, FOS, FUT6, GSK3B, Grb10, HES6, HLA-G, HNRPAB, HOXB13, HSPE1, KIF22, KIFC1, KLRK1, Ki-67, LAT, LMYC, MAD2L1, MSH2, MSH3, NR4A1, PDGFA, PRDX2, RAB32, RAD54L, RANBP2, RCC1, ROCK2, RhoB, S100P, SAT, SOD1, SOS1, STK15, TCF-1, TOP2A, TP53BP1, UBE2C, VCP, and cMYC, or their corresponding expression, wherein an increased expression of one or more of the genes, is positively correlated with an increased likelihood of a positive response to chemotherapy.

### SUMMARY OF THE INVENTION

The present inventors demonstrate herein a significant and novel departure from previous findings regarding the expression levels of MSH3 in assessing prognosis and/or predicting the response of cancer to chemotherapy in colorectal cancer.

In one embodiment the present invention provides a method for treating a patient at risk for or diagnosed with one or more adenocarcinomas, the method comprising: determining the overall expression of MSH3 in cells suspected of being adenocarcinoma cells obtained from the patient and predicting the efficacy of therapy with an anti-neoplastic agent for treating the patient, wherein a decrease in the overall expression of MSH3 in the patient cells when compared to the expression of MSH3 in normal cells indicates a predisposition to responsiveness to anti-neoplastic agent therapy, wherein the therapy comprises administering an effective amount of the anti-neoplastic agent to the patient. The adenocarcinomas described hereinabove are selected from the group consisting of colorectal cancer (CRC), lung cancer, cervical cancer, ovarian cancer, prostate cancer, kidney cancer, liver cancer, testicular cancer, bladder cancer, vaginal cancer, breast cancer, esophageal cancer, pancreatic cancer, and stomach cancer. In more specific aspects the adenocarcinoma is CRC and the adenocarcinoma comprises a solid tumor.

In another aspect the step of determining the overall level of expression of MSH3 comprises analyzing cells suspected of being adenocarcinoma cells for MSH3 protein expression, MSH3 nucleic acid expression or both. In another aspect the step of determining the overall level of expression of MSH3 comprises performing mass spectrometry analysis of MSH3 nucleic acids obtained from the individual. In yet another aspect the step of determining the overall level of expression of MSH3 comprises rolling circle amplification of a portion of a MSH3 nucleic acid obtained from the individual. In another aspect the step of determining the overall level of expression of MSH3 comprises hybridization with an allele specific probe, an antibody probe or both. In another aspect the step of determining the overall level of expression of MSH3 comprises immunohistochemistry.

In a related aspect the anti-neoplastic agent is selected from the group consisting of 1,3-bis(2-chloroethyl)-1-nitrosourea, busulfan, carmustine, chlorambucil, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, temozolomide, topotecan, and ionizing radiation. In one aspect the anti-neoplastic agent is an interstrand crosslinking agent. In another aspect the anti-neoplastic agent is an interstrand crosslinking agent selected from cisplatin, carboplatin, oxaliplatin, furocoumarins, or psoralen. In yet another aspect the anti-neoplastic agent is a poly (ADP-ribose) polymerase (PARP) inhibitor selected from the group consisting of olaparib, isoindolinone derivatives, veliparib, iniparib, and 4-methoxy-carbazole derivatives.

Another embodiment of the present invention provides a method for treating a patient at risk for or diagnosed with an adenocarcinoma, the method comprising: (i) determining the overall expression of MSH3 in the cells suspected of being adenocarcinoma cells obtained from the patient and (ii) predicting the efficacy of therapy with an anti-neoplastic agent for treating the patient, wherein a decrease in the overall expression of MSH3 in the patient cells when compared to the expression of MSH3 in normal cells indicates a predisposition to responsiveness to anti-neoplastic agent therapy, wherein the therapy comprises administering an effective amount of the anti-neoplastic agent therapy to the patient. In one aspect the adenocarcinomas are selected from the group consisting of colorectal cancer (CRC), lung cancer, cervical cancer, ovarian cancer, prostate cancer, kidney cancer, liver cancer, testicular cancer, bladder cancer, vaginal cancer, breast cancer, esophageal cancer, pancreatic cancer, and stomach cancer. In another aspect the adenocarcinoma is CRC. In yet another aspect the adenocarcinoma comprises a solid tumor.

The step of determining the overall level of expression of MSH3 as described hereinabove comprises analyzing the cells suspected of being adenocarcinoma cells for MSH3 protein expression, MSH3 nucleic acid expression or both. In one aspect the step of determining the overall level of expression of MSH3 comprises performing mass spectrometry analysis of MSH3 nucleic acids obtained from the individual. In another aspect the step of determining the overall level of expression of MSH3 comprises rolling circle amplification of a portion of a MSH3 nucleic acid obtained from the individual. In yet another aspect the step of determining the overall level of expression of MSH3 comprises hybridization with an allele specific probe, an antibody probe or both. In another aspect the step of determining the overall level of expression of MSH3 comprises immunohistochemistry.

In one aspect the anti-neoplastic agent is selected from the group consisting of 1,3-bis(2-chloroethyl)-1-nitrosourea, busulfan, carmustine, chlorambucil, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, temozolomide, topotecan, and ionizing radiation. In another aspect the anti-neoplastic agent is an interstrand crosslinking agent. In another aspect the anti-neoplastic agent is an interstrand crosslinking agent selected from cisplatin, carboplatin, oxaliplatin, furocoumarins, or psoralen. In yet another aspect the anti-neoplastic agent is a poly (ADP-ribose) polymerase (PARP) inhibitor selected from the group consisting of olaparib, isoindolinone derivatives, veliparib, iniparib, and 4-methoxy-carbazole derivatives.

In yet another embodiment the present invention discloses a method for treating a patient at risk for or diagnosed with colorectal cancer, the method comprising: determining the overall expression of MSH3 in cells suspected of being colorectal cancer cells from the patient and predicting the efficacy of therapy with an anti-neoplastic agent for treating the patient, wherein a decrease in the overall expression of MSH3 in the patient cells when compared to the expression of MSH3 in normal colorectal cells indicates a predisposition to responsiveness to anti-neoplastic agent therapy, wherein the therapy comprises administering an effective amount of the anti-neoplastic agent therapy to the patient.

In one aspect the step of determining the overall level of expression of MSH3 comprises analyzing a tissue sample suspected of being colorectal cancer for MSH3 protein expression. In another aspect the step of determining the overall level of expression of MSH3 comprises analyzing a tissue sample suspected of being colorectal cancer for MSH3 nucleic acid expression. In yet another aspect the step of determining the overall level of expression of MSH3 comprises performing mass spectrometry analysis of MSH3 nucleic acids obtained from the individual. In another aspect the step of determining the overall level of expression of MSH3 comprises rolling circle amplification of a portion of a MSH3 nucleic acid obtained from the individual. In another aspect the step of determining the overall level of expression of MSH3 comprises hybridization with an allele specific probe, an antibody probe or both. In yet another aspect the step of determining the overall level of expression of MSH3 comprises immunohistochemistry.

In one aspect the anti-neoplastic agent is selected from the group consisting of 1,3-bis(2-chloroethyl)-1-nitrosourea, busulfan, carmustine, chlorambucil, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, temozolomide, topotecan, and ionizing radiation. In a specific aspect the anti-neoplastic agent is an interstrand crosslinking agent. In another aspect the anti-neoplastic agent is an interstrand crosslinking agent selected from cisplatin, carboplatin, oxaliplatin, furocoumarins, or psoralen. In yet another aspect the anti-neoplastic agent is a poly (ADP-ribose) polymerase (PARP) inhibitor selected from the group consisting of olaparib, isoindolinone derivatives, veliparib, iniparib, and 4-methoxy-carbazole derivatives.

The present invention further provides a method for selecting a cancer therapy for a patient at risk for or diagnosed with colorectal cancer, the method comprising the step of determining the overall expression level of MSH3 of the patient and predicting the efficacy of therapy with a anti-neoplastic agent for treating the patient with an anti-neoplastic agent, wherein a decrease in the overall level of expression of MSH3 indicates that the DNA crosslinking agent is a suitable therapy for the patient. In one aspect the step of determining the overall level of expression of MSH3 comprises analyzing a tissue sample suspected of being colorectal cancer for MSH3 protein expression. In another aspect the step of determining the overall level of expression of MSH3 comprises analyzing a tissue sample suspected of being colorectal cancer for MSH3 nucleic acid expression. In another aspect the step of determining the overall level of expression of MSH3 comprises performing mass spectrometry analysis of MSH3 nucleic acids obtained from the individual. In yet another aspect of the method described hereinabove the step of determining the overall level of expression of MSH3 comprises rolling circle amplification of a portion of a MSH3 nucleic acid obtained from the individual. In a related aspect of the method the step of determining the overall level of expression of MSH3 comprises hybridization with an allele specific probe, antibody probe, or immunohistochemistry. In a specific aspect of the method the anti-neoplastic is an interstrand crosslinking agent. In another aspect the anti-neoplastic is selected from cisplatin, carboplatin, oxaliplatin, furocoumarins, or psoralen. In another aspect the anti-neoplastic agent is a poly (ADP-ribose) polymerase (PARP) inhibitor selected from the group consisting of olaparib, isoindolinone derivatives, veliparib, iniparib, and 4-methoxy-carbazole derivatives.

Another embodiment disclosed herein relates to a method for stratifying a patient in a subgroup of a clinical trial of a cancer therapy, the method comprising: determining the overall expression of MSH3 in cells suspected of being cancer cells from the patient and predicting the efficacy of therapy with a candidate drug for treating the patient, wherein a decrease in the overall expression of MSH3 in the patient cells when compared to the expression of MSH3 in normal cells indicates a predisposition to responsiveness to therapy with the candidate drug, wherein the therapy comprises administering an effective amount of the candidate drug to patients and the level of expression of MSH3 enables the stratification of the patient into a subgroup for the clinical trial. In one aspect the cancer cells are selected from the group consisting of colorectal cancer (CRC), lung cancer, cervical cancer, ovarian cancer, prostate cancer, kidney cancer, liver cancer, testicular cancer, bladder cancer, vaginal cancer, breast cancer, esophageal cancer, pancreatic cancer, and stomach cancer. In specific aspects the cancer cells are colorectal cancer cells and the cancer cells are in a solid tumor.

In one aspect of the method the step of determining the overall level of expression of MSH3 comprises analyzing a tissue sample suspected of being colorectal cancer for MSH3 protein expression, MSH3 nucleic acid expression or both. In another aspect the step of determining the overall level of expression of MSH3 comprises performing mass spectrometry analysis of MSH3 nucleic acids obtained from the individual. In another aspect the step of determining the overall level of expression of MSH3 comprises rolling circle amplification of a portion of a MSH3 nucleic acid obtained from the individual. In yet another aspect the step of determining the overall level of expression of MSH3 comprises hybridization with an allele specific probe or an antibody probe. In another aspect the step of determining the overall level of expression of MSH3 comprises immunohistochemistry. In a related aspect the candidate agent is a genotoxic agent or a poly (ADP-ribose) polymerase (PARP) inhibitor.

In yet another embodiment the present invention describes steps for stratifying a patient in a subgroup of colorectal cancer by a method comprising the steps of: determining the overall expression of MSH3 in cells suspected of being colorectal cancer cells from the patient and predicting the stage of the colorectal, wherein a decrease in the overall expression of MSH3 in the patient cells when compared to the expression of MSH3 in normal colorectal cells disease progression. In one aspect of the stratification method discloses above the disease progression and a decrease in MSH3 expression indicates a predisposition of the colorectal cancer to an anti-neoplastic agent therapy.

The present invention further discloses a method for treating a patient at risk for or diagnosed with colorectal cancer, the method comprising the steps of: (i) determining the overall expression of MSH3 in cells suspected of being colorectal cancer cells from the patient which indicates a predisposition to responsiveness to therapy with one or more DNA crosslinking agents, (ii) determining a continued decrease in the overall expression of MSH3 in the patient, and (iii) administering a therapeutically effective amount of a DNA crosslinking agent in an amount sufficient to eliminate colorectal cancer cells with decreases MSH3 expression.

In one embodiment the present invention relates to a method of performing a clinical trial to evaluate a candidate drug believed to be useful in treating a disease state associated with MSH3 gene expression, the method comprising: a) measuring the level of MSH3 expression from tissue suspected of having colorectal cancer from a set of patients, b) administering a candidate drug to a first subset of the patients, and a placebo to a second subset of the patients, c) repeating step a) after the administration of the candidate drug or the placebo, and d) determining if the candidate drug reduces the number of colorectal cells that have a decrease in the expression of MSH3 that is statistically significant as compared to any reduction occurring in the second subset of patients, wherein a statistically significant reduction indicates that the candidate drug is useful in treating said disease state.

In another embodiment the present invention provides a method for determining whether a mammalian colorectal cancer is likely to be resistant or responsive to a DNA damaging agent for the treatment of colorectal cancer, the method comprising the step(s) of: examining a biological sample from the cancer for a decrease in the overall expression of MSH3 and identifying the colorectal cancer as having an enhanced susceptibility to the DNA damaging agent where there is decreased expression or activity of MSH3 relative to the same biomarker's expression or activity level in the cancer that is responsive to the DNA damaging agent.

In yet another embodiment the present invention discloses a biomarker for colorectal cancer disease progression, wherein the biomarker is MSH3 and a decrease in the overall expression of MSH3 in colorectal cancer cells obtained from a patient is indicative of colorectal cancer disease progression when compared to MSH3 expression is normal colorectal cancer cells or colorectal cancer cells obtained at an earlier time-point from the same patient. In one aspect the overall level of expression of MSH3 comprises analyzing a tissue sample suspected of being colorectal cancer for MSH3 protein expression. In another aspect the overall level of expression of MSH3 comprises analyzing a tissue sample suspected of being colorectal cancer for MSH3 nucleic acid expression. In another aspect the overall level of expression of MSH3 comprises performing mass spectrometry analysis of MSH3 nucleic acids obtained from the individual. In yet another aspect the overall level of expression of MSH3 comprises rolling circle amplification of a portion of a MSH3 nucleic acid obtained from the individual. In another aspect the overall level of expression of MSH3 comprises hybridization with an allele specific probe, antibody probe or by immunohistochemistry.

The present invention further describes a kit for a diagnosis of colorectal cancer comprising biomarker detecting reagents for determining a differential expression level of MSH3 and instructions for their use in diagnosing risk for colorectal cancer. In one aspect both MSH3 mRNA and protein expression levels in a sample from a patient at risk for colorectal is significantly decreased compared to that of a normal subject. In another aspect the MSH3 mRNA expression level is decreased in the patient at risk for colorectal cancer in comparison a normal subject. In yet another aspect the MSH3 protein expression level is decreased in the patient as at risk for colorectal cancer in comparison to a normal subject.

Finally, the present invention provides a method for diagnosing or detecting colorectal cancer progression in a human subject comprising the steps of: (i) identifying the human subject suspected of suffering from colorectal cancer, (ii) obtaining one or more biological samples from the subject, wherein the biological samples are selected from the group consisting of a tissue sample, a fecal sample, a cell homogenate, and one or more biological fluids comprising, (iii) measuring an overall expression pattern of MSH3 in one or more cells obtained from the biological samples of the subject, and (iv) comparing the overall expression pattern of the MSH3 from the biological sample of the subject suspected of suffering from colorectal cancer with the overall expression pattern of MSH3 from a biological sample of a normal subject, wherein the normal subject is a healthy subject not suffering from colorectal cancer, wherein a decrease in the overall expression pattern of the MSH3 in the biological sample of the subject is indicative of the presence, risk for developing or both of colorectal cancer.

In one aspect of the diagnostic method disclosed hereinabove a significant decrease in the expression levels of MSH3 mRNA, MSH3 protein or both, are indicative of the presence, risk for developing or both of invasive colorectal cancer. In another aspect the step of determining the overall level of expression of MSH3 comprises analyzing the one or more cells from the biological sample for MSH3 nucleic acid expression. In another aspect the step of determining the overall level of expression of MSH3 comprises performing mass spectrometry analysis of MSH3 nucleic acids obtained from the subject. In yet another aspect the step of determining the overall level of expression of MSH3 comprises performing a rolling circle amplification of a portion of a MSH3 nucleic acid obtained from the subject. In one aspect step of determining the overall level of expression of MSH3 comprises hybridization with an allele specific probe or an antibody probe. In another aspect the step of determining the overall level of expression of MSH3 comprises immunohistochemistry. In yet another aspect the method is used for treating a patient at risk or suffering from colorectal cancer, selecting a DNA crosslinking agent therapy for a patient at risk or suffering from colorectal cancer, stratifying a patient in a subgroup of colorectal cancer or for a colorectal cancer therapy clinical trial, determining resistivity or responsiveness to a colorectal cancer therapeutic regimen, developing a kit for diagnosis of colorectal cancer or any combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 shows the MSH3 expression of the HCT116+3+5-derived clones stably transfected with MSH3 shRNA is controlled by a tet-off system: (1A) Western blot analysis of MSH3, MLH1 and β-actin in HCT116, HCT116+3, HCT116+3+5, and the three HCT116+3+5-derived clones, G1, G2 and G5 cells, (1B) Western blot analysis of MSH3 and β-actin in HCT116+3+5, G1, G2 and G5 cells cultured in the medium with and without 1 μg/ml of doxycycline. Relative MSH3 expression was calculated by densitometry and the results were obtained from three or more independent studies.

FIG. 2 shows that MSH3-deficient cells are more sensitive to cisplatin and oxaliplatin than MSH3-proficient cells: (2A) Clonogenic survival fraction of HCT116, HCT116+3, HCT116+3+5 and G5 cells treated with cisplatin, (2B) Clonogenic survival fraction of G5 cells cultured with and without doxycycline 1 μg/ml, which were treated with cisplatin, (2C) Clonogenic survival fraction of HCT116, HCT116+3, HCT116+3+5 and G5 cells treated with oxaliplatin, (2D) Clonogenic survival fraction of G1, G2 and G5 cells cultured with and without doxycycline 1 μg/ml, which were also treated with oxaliplatin, (2E) Decrease in S-phase population, and (2F) Increase in sub-G1 population of the HCT116+3+5 and G1 cells, (2G) Decrease in relative BrdU incorporation compared to non-treated controls, and (2H) Increase in anti-active caspase-3 positive cells in immunofluorescence in the HCT116+3+5 and G5 cells. Data are represented as mean±standard error of mean (SE) from three or more independent studies. The statistical difference was determined by two-sided Student's t test. The asterisks *, ** and *** represent p<0.05, p<0.01 and p<0.001, respectively. NS represents p=0.05 or more. Representative data from one of the three MSH3-deficient clones is shown in this figure.

FIG. 3 shows the transient depletion of MSH3 by siRNA also sensitizes HCT116+3+5 cells to cisplatin and oxaliplatin: (3A) Western blot analysis of MSH3 and β-actin in MSH3-depleted HCT116+3+5 cells by transient siRNA transfection. Comparison of clonogenic survival fraction of HCT116+3+5 cell lines treated with cisplatin, (3B) and oxaliplatin, (3C) after transfection between with non-targeted (control) siRNA and with MSH3 siRNA, (3D) Western blot analysis of MSH3 and β-actin in HT29 cells treated with non-targeted siRNA and with MSH3 siRNA. Cells were extracted 72 hours after siRNA transfection. Comparison of clonogenic survival fractions of HT29 cells treated with cisplatin (3E) and oxaliplatin (3F) after transfection with control siRNA and MSH3 siRNA. Data are represented as mean±SE from five independent experiments. The statistical difference was determined by two-sided Student's-t test. The asterisks * and ** represent p<0.05 and p<0.01, respectively; NS represents p>0.05.

FIG. 4 shows the transient depletion of MLH1 by siRNA does not affect the resistance to cisplatin and oxaliplatin of the MSH3-proficient and -deficient cells: (4A). Western blot analysis of MLH1 and β-actin in MLH1-depleted HCT116+3+5 and G5 cells following transient siRNA transfection, (4B) Clonogenic survival fraction of HCT116+3+5 and G5 cells treated with N-methyl-N′-nitro-N-nitrosoguanidine after transfection with control siRNA or MLH1 siRNA. Comparison of the clonogenic survival fraction between MLH1-depleted HCT116+3+5 and G5 cells treated with cisplatin (4C) and oxaliplatin (4D). Data are represented as mean±SE from four or more independent experiments. The statistical difference was determined by two-sided Student's t test. The asterisks * and ** represent p<0.05 and p<0.01, respectively; NS represents p>0.05.

FIG. 5 demonstrates that MSH3-deficient cells show a decrease in DNA double strand break repair efficiency: (5A) Immunofluorescence staining for pH2AX foci formation in the HCT116+3+5, G5 with doxycycline and G5 cells without doxycycline. The cells were treated with 5 μM of oxaliplatin treatment for 6 hours, and were analyzed by immunofluorescence after the indicated hours. upper panel; DAPI, lower panel: pH2AX, (5B) Inefficient decline of pH2AX positive cells in the MSH3-deficient cells, (5C) Immunofluorescence staining for 53BP1 foci formation in G5 with doxycycline and G5 cells without doxycycline. The cells were treated with 5 μM of oxaliplatin treatment for 6 hours, and were analyzed by immunofluorescence after 48 hours (5D). At least 100 cells were counted in each slide. Data are represented as mean±SE from three or four independent experiments. The statistical difference between MSH3-deficient and -proficient G5 was determined by two-sided Student's t test. The asterisks * and *** represents p<0.05 and p<0.001, respectively.

FIG. 6 shows that MSH3-deficient cells are sensitive to olaparib, a PARP inhibitor, and the combination with oxaliplatin: (6A) Clonogenic survival of HCT116+3+5, G5 without doxycycline and G5 cells with doxycycline, which were treated with 2 μM of oxaliplatin, 2 μM of olaparib and the combination of these two drugs and (6B) Clonogenic survival of HT29 cells, which were treated with 1 μM of oxaliplatin, 2 μM of olaparib and the combination of these two drugs. Data are represented as mean±SE from three or more independent experiments. The statistical difference was determined by two-sided Student's t-test. The asterisks *, **, and *** represent p<0.05, p<0.01 and p<0.001, respectively.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The abbreviations used herein include: MMR, mismatch repair; MSI, microsatellite instability; CRC, colorectal cancer; MeG, O6-methylguanine; ICLs, interstrand crosslinks; NER, nucleotide excision repair; HR, homologous recombination; PARP, poly(ADP-ribose) polymerase; EMAST, elevated microsatellite alterations at tetranucleotide repeats; DSB, double strand break; BrdU, bromodeoxyuridine; pH2AX, phosphorylated H2AX.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions which are characterized by cancer of cells of the duodenum and small intestine (jejunum and ileum).

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular. The term “stool” as used herein is a clinical term that refers to feces excreted by humans.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2nd Ed. Cold Spring Harbor Press (1989) which is hereby incorporated by reference in its entirety for all purposes above.

The term “rolling circle amplification (RCA)” as used herein describes a method of DNA replication and amplification that results in a strand of nucleic acid comprising one or more copies of a sequence that is a complimentary to a sequence of the original circular DNA. This process for amplifying (generating complimentary copies) comprises hybridizing an oligonucleotide primer to the circular target DNA, followed by isothermal cycling (e.g., in the presence of a ligase and a DNA polymerase). A single round of amplification using RCA results in a large amplification of the sequences in the circular target to obtain a high concentration the desired oligonucleotide on a single strand of nucleic acid. Because the desired nucleic acid sequence becomes the predominant sequence (in terms of concentration) in the mixture, it is said to be “RCA amplified”. With RCA, it is possible to amplify a single copy of a particular nucleic acid sequence to a level detectable by several different methodologies (e.g., hybridization with a labeled probe; incorporation of biotinylated primers followed by avidin-enzyme conjugate detection; incorporation of 32 P-labeled deoxynucleotide triphosphates, such as dCTP or dATP, into the amplified segment)

As used herein the term “antibody probe” refers to an antibody that is specific for and binds to any target antigen. Such a target antigen may be a peptide, protein, carbohydrate or any other biopolymer to which an antibody will bind with specificity.

The term “biomarker” as used herein in various embodiments refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH2Cl2, elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me2S (source: infection; diagnosis: trench mouth).

As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

As used herein, the term “clinical trial” includes any research study designed to collect clinical data on responses to a particular treatment, and includes but is not limited to phase I, phase II and phase III clinical trials. Standard methods are used to define the patient population and to enroll subjects.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The term “candidate drug” as used herein refers to any compound, of whatever origin, suitable for being screened for its activity in reducing the number of colorectal cells that have decreased MSH3 expression according to the methods of the present invention.

The term “genotoxic agent” as used herein is defined to include both chemical and physical agents capable of causing damage to human DNA or the gene. Carcinogens and mutagens are common examples of chemical genotoxic agents, while UV radiation, γ and X-rays and the like when they produce oxidized DNA product are common examples of physical genotoxic agents.

The term “anti-neoplastic agent” refers to agents that have the functional property of inhibiting the development or progression of a neoplasm in a mammal, e.g., a human, and may also refer to the inhibition of metastasis or metastatic potential.

The term “kit” or “testing kit” denotes combinations of reagents and adjuvants required for an analysis. Although a test kit consists in most cases of several units, one-piece analysis elements are also available, which must likewise be regarded as testing kits.

MSH3 gene (Accession No. P20585) is one of the DNA mismatch repair (MMR) genes that has undergone somatic mutation frequently in MMR-deficient cancers. MSH3, together with MSH2 forms the MutSβ heteroduplex, which interacts with interstrand crosslinks (ICLs) induced by drugs such as cisplatin and psoralen. However, the precise role of MSH3 in mediating the cytotoxic effects of ICL-inducing agents remains poorly understood. The present inventors demonstrate herein the effects of MSH3 deficiency on cytotoxicity caused by cisplatin and oxaliplatin, another ICL-inducing platinum drug.

Using isogenic HCT116-derived clones in which MSH3 expression is controlled by shRNA expression in a tet-off system, it was discovered that MSH3 deficiency sensitized cells to both cisplatin and oxaliplatin at clinically relevant doses. Interestingly, siRNA-induced down-regulation of the MLH1 protein did not affect MSH3-dependent toxicity of these drugs, indicating that this process does not require participation of the canonical MMR pathway.

Furthermore, MSH3-deficient cells maintained higher levels of phosphorylated H2AX and 53BP1 after oxaliplatin treatment in comparison to MSH3-proficient cells, suggesting that MSH3 plays an important role in repairing DNA double strand breaks (DSB). This role of MSH3 was further supported by the findings herein that MSH3-deficient cells were sensitive to olaparib, a Poly(ADP-ribose) polymerase inhibitor. Moreover, the combination of oxaliplatin and olaparib exhibited a synergistic effect compared to either treatment individually. Collectively, these results demonstrate that MSH3 deficiency contributes to the cytotoxicity of platinum drugs through deficient DSB repair. These data allow for effective prediction and treatments for cancers with MSH3 deficiency.

The findings of the present invention represent a significant and novel departure from previous findings regarding genes (including MSH3) and gene sets useful in assessing prognosis and/or predicting the response of cancer to chemotherapy. U.S. Patent Publication No. 20090305277 filed by Baker et al., describes a method of predicting a likelihood that a human patient diagnosed with cancer based on determining an expression level of at least one gene that is the opposite of the present invention, namely that an increase in the expression of certain genes, including, MSH3, are positively correlated with an increased likelihood of a positive response to chemotherapy. However, the application includes no cellular or tissue data, but rather, uses a generic gene mining approach to reach their conclusions.

The DNA mismatch repair (MMR) system is composed of several proteins such as MLH1, MSH2, MSH6, MSH3 and PMS2, eliminates replication errors and maintains genomic stability. MutSα, a MSH2/MSH6 heterodimer, recognizes single base mismatches, whereas MutSβ, a MSH2/MSH3 heterodimer, primarily recognizes 2-4 bp insertion-deletion loops (1,2). The MutL complex, mainly MutLα, a MLH1/PMS2 heterodimer, forms a ternary complex with a MutS heterodimer that binds to mismatches to DNA mismatches during replication, and leads to recruitment of other proteins to complete the process of DNA MMR. Germline mutations in MMR genes result in Lynch syndrome, which is characterized by hereditary predisposition to cancers with microsatellite instability (MSI) in the colon, endometrium, ovaries and urinary tract (3,4). In contrast, MMR deficiency resulting from MLH1 promoter methylation causes sporadic MSI tumors, including colorectal cancer (CRC) (˜15%), endometrial cancer (20-25%) and ovarian cancer (˜12%) (4-6).

The MMR system also participates in repairing certain DNA adducts generated by DNA damaging agents such as alkylating agents and 6-thioguanine. The primary cytotoxic lesion generated by alkylating agents is O⁶-methylguanine (^{Me}G), which causes ^{Me}G-C or ^{Me}G-T mispairs (7). MutSα recognizes these mispairs and recruits MutLα for the subsequent repair reactions (8,9). Loss of MutSα or MutLα renders a cell tolerant to the cytotoxic effects of these drugs, suggesting that these two complexes are also linked to a signal transduction pathway which leads to cell growth arrest or cell death (10,11).

On the other hand, MutSβ recognizes interstrand crosslinks (ICLs) generated by DNA crosslinkers such as psoralen and cisplatin. MutSβ is involved in the recognition and uncoupling of the psoralen-induced ICLs in mammalian cell extracts (12). Recently, it has been shown that MutSβ interacts with XPA-RPA or XPC-RAD23B, both of which are involved in nucleotide excision repair (NER), in the recognition of psoralen ICLs and promotes the NER process (13,14). The level of homologous recombination (HR) that repairs ICLs is also dependent on MutSβ but not on MutSα or MLH1. These results suggest that MutSβ may cooperate with the NER, HR and Fanconi anemia proteins for repairing psoralen-induced ICLs (15). In addition, MutSβ also binds to cisplatin-induced ICLs together with PARP-1, DNA ligase III, XRCC-1, Ku80 and Ku70, suggesting that MutSβ may also cooperate with other repair pathways to recognize and repair platinum drug-induced ICLs (16).

Oxaliplatin, a third generation platinum drug, is one of the key drugs that are currently being used for the treatment with CRC patients. Similar to cisplatin, oxaliplatin also forms intrastrand crosslinks and ICLs (17). However, the detailed molecular mechanisms involved in repair and the cytotoxic effects of oxaliplatin-induced adducts, especially ICLs, have not been extensively explored.

Considering that MutSβ complex plays a role in repairing ICLs, the present inventors recognized that MSH3-deficiency may halt the repair of ICLs induced by platinum drugs, resulting in enhanced cytotoxicity of these drugs in cancer patients. Additionally, because MSH3-deficiency results in suppressed HR (15) and HR-defective cells are hypersensitive to Poly(ADP-ribose) polymerase (PARP) inhibitors (18,19), the present inventors further recognized that MSH3-deficiency may also result in sensitization of cells to PARP inhibitors. In MSI CRC, frequent frameshift mutations (20-50%) within the mononucleotide [A]₈ repeats in exon 7 of MSH3 results in loss or reduction of MSH3 (20-22). Recently, the present inventors found that the MSH3-negative cancer cell population exists within sporadic CRC tissues that exhibit low levels of MSI and/or elevated microsatellite alterations at tetranucleotide repeats (EMAST) (23). If MSH3 deficiency dictates the toxicity of platinum drugs and PARP inhibitors in a clinical setting, MSH3 status can be used as a predictive marker for the chemotherapeutic outcome in patients with MSH3-deficient cancers. To demonstrate that MSH3 status can be used as a predictive marker for the chemotherapeutic outcome in patients with MSH3-deficient cancers, the present inventors used isogenic cell lines in which MSH3 protein expression can be regulated thorough shRNA expression in a tet-off system, and investigated the effect of MSH3 deficiency on the cellular sensitivity to two platinum drugs and a well-known PARP inhibitor. These studies uncovered novel molecular evidence that MSH3 deficiency in CRC cell lines contributes to the cytotoxicity of platinum drugs, especially as a result of compromised double strand break (DSB) repair.

Reagents—Cisplatin, oxaliplatin, N-Methyl-N-Nitro-N-Nitrosoguanidine (MNNG) and propidium iodide were purchased from Sigma-Aldrich (St. Louis, Mo.). Olaparib, a PARP inhibitor, was purchased from Selleck Chemicals (Houston, Tex.).

Cell lines and cell culture—The human colon cancer cell lines HCT116, HCT116+ch.3 (HCT116+3), HCT116+ch.3+ch.5 (HCT116+3+5) have been described previously (10,23). HCT116+3+5 cells were stably transfected with a tetracycline-regulated retroviral vector, the TMP (Open Biosystems, Huntsville, Ala.) that encodes shRNA against MSH3. Stable MSH3-deficient clones G1, G2 and G5 were isolated (see results and (23)). HCT116, HCT116+3, and HCT116+3+5 cells were grown in IMDM (Invitrogen, Rockville, Md.) with 10% fetal bovine serum. The G1, G2 and G5 cells were maintained in IMDM with 10% fetal bovine serum and 0.6 μg/ml of puromycin. To turn off the expression of MSH3 shRNA, 1 μg/ml of doxycycline was added to the culture medium.

Western blot analysis—Proteins from cell lysates were prepared, separated on the SDS-PAGE and transferred to PVDF membranes as described previously (31). Anti-hMSH3 mouse monoclonal antibody (dilution; 1:250, Clone 52, BD Pharmingen, San Jose, Calif.), anti-hMLH1 mouse monoclonal antibody (1:200, G168-728, BD Pharmingen) and anti-β-actin antibody (1:10000, Clone AC-15, Sigma-Aldrich) were used as primary antibodies for the detection of specific proteins. Goat anti-mouse antibody (1:3000, sc-2005, Santa Cruz Biotechnology, Santa Cruz, Calif.) was used as a secondary antibody. The signal amplification and detection was achieved by exposing the membrane to ECL reagent (GE Healthcare, Piscataway, N.J.), followed by visualization on the Storm imaging system (Amersham, Piscataway, N.J.).

Clonogenic survival assay—Two hundred cells were seeded in each well of a six-well plate. For the measurement of the cytotoxicity caused by cisplatin or oxaliplatin, the cells were treated with the drugs for 24 hours once the cells were attached to the plate. For the measurement of the cytotoxicity caused by olaparib, cells were continuously treated with the drug during the experiments. After 8-10 days, the number of colonies (colonies with >50 cells) were counted, and the relative change in clonogenic survival of drug-treated versus untreated cells was determined.

Cell cycle analysis—One million cells were seeded in 10 cm plates. Once attached, the cell lines were treated with oxaliplatin for 24 hours. After an additional 48 hours, cells were washed twice with cold PBS, fixed in cold 70% ethanol at −20° C. overnight or for several days. The ethanol fixed cells (2×10⁶) were subsequently washed with PBS twice and incubated with 300 μl of PBS and 0.15% RNase A for 15 minutes at 37° C. The cells were stained with 75 μg/ml propidium iodide for 30 minutes and then analyzed for DNA content using the FACSCantoII flow cytometer (BD Biosciences, San Jose, Calif.). Cell cycle data was analyzed by the Flowjo software (Tree Star, Ashland, Oreg.).

Proliferation assay—The proliferation index was measured by bromodeoxyuridine (BrdU) incorporation in HCT116+3+5 and G5 cells, 48 hours after the initial 24 hour treatment with cisplatin or oxaliplatin (Cell Proliferation ELISA, BrdU, Roche Diagnositics, Indianapolis, Ind.). Experiments were performed in triplicate and data was obtained from three or four independent experiments.

siRNA treatment—MLH1 siRNA, MSH3 siRNA and non-targeted siRNA were purchased from Dharmacon (Lafayette, Colo.). Two hundred thousand cells were seeded in 24-well plates. After overnight incubation, the cells were transfected with 83 nM of the targeted siRNAs or non-targeted siRNAs using Lipofectamine 2000 (Invitrogen, Carlsbad, Calif.) according to the manufacturer's instructions. Two days after transfection, the cells were harvested and re-plated for clonogenic survival assays.

Immunofluorescence staining—Ten thousand cells were grown on glass coverslips in a 12-well plate. The cells were fixed with 4% paraformaldehyde (pH 7.5) in PBS for 15 minutes, permeabilized with 0.3% Triton X-100 for five minutes, and then blocked with 10% goat serum (Invitrogen, Carlsbad, Calif.) for one hour. The cells were subsequently incubated with an anti-active caspase-3 antibody (1:500, G748, Promega, Madison, Wis.) an anti-phosphorylated H2AX (pH2AX) antibody (1:5000, JBW301, Millipore Corporation, Billerica, Mass.), or an anti-53BP1 antibody (1:600, ab21083, Abcam, Cambridge, Mass.) for one hour, followed by a secondary antibody (1:800, Alexa Fluor 555 goat anti-mouse or anti-rabbit antibody, Invitrogen) for 40 minutes. Prolong Gold with DAPI (Invitrogen) was used in mounting the medium. The images were obtained using AxioSkop2 multichannel epifluorescence microscope equipped with the AxioVision software (Carl Zeiss, Thornwood, N.Y.).

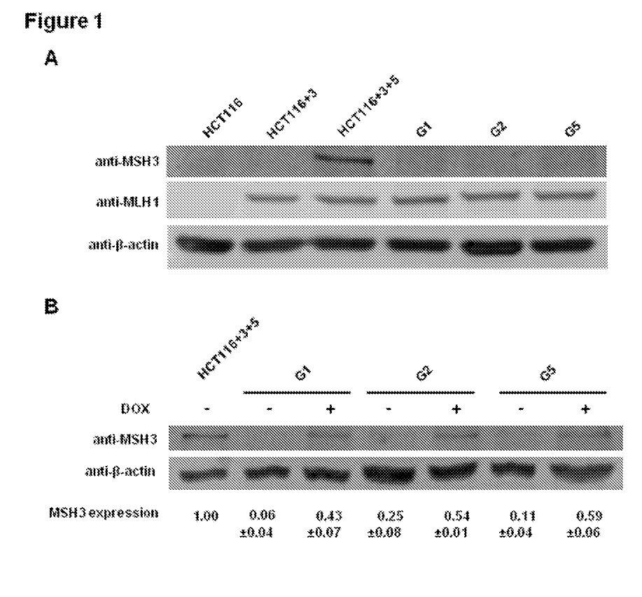

MSH3 expression is controlled by doxycycline in MSH3-deficient clones. The present inventors first determined whether MSH3 expression in G1, G2 and G5 cell clones of HCT116 CRC cells is controlled by doxycycline. HCT116 and HCT116+3 cells were used as negative controls and HCT116+3+5 as positive control for MSH3 expression. HCT116 and HCT116+3 cells showed no detectable MSH3 protein expression (FIG. 1A), which is consistent with HCT116 cells harboring homozygous frameshift mutations in a mononucleotide repeat of the MSH3 exon 7 (23). HCT116+3+5, generated from MSH3-deficient HCT116+3 by transfer of a copy of chromosome 5, showed MSH3 expression. While no MSH3 was detected in G1, G2 and G5 clones in the absence of doxycycline, addition of doxycycline restored MSH3 expression in all of these clones to about 40-60% of the levels of parental HCT116+3+5 (FIGS. 1A & 1B). While it may be technically challenging to expect complete blockade for the production of MSH3 shRNA in these cell lines even in the presence of doxycycline, the protein level in these results was enough to analyze the effect of MSH3 on drug sensitivity in this study because this level of MSH3 in G5 cells is enough to recover MSH3 functions regarding EMAST phenotype in vitro (23).

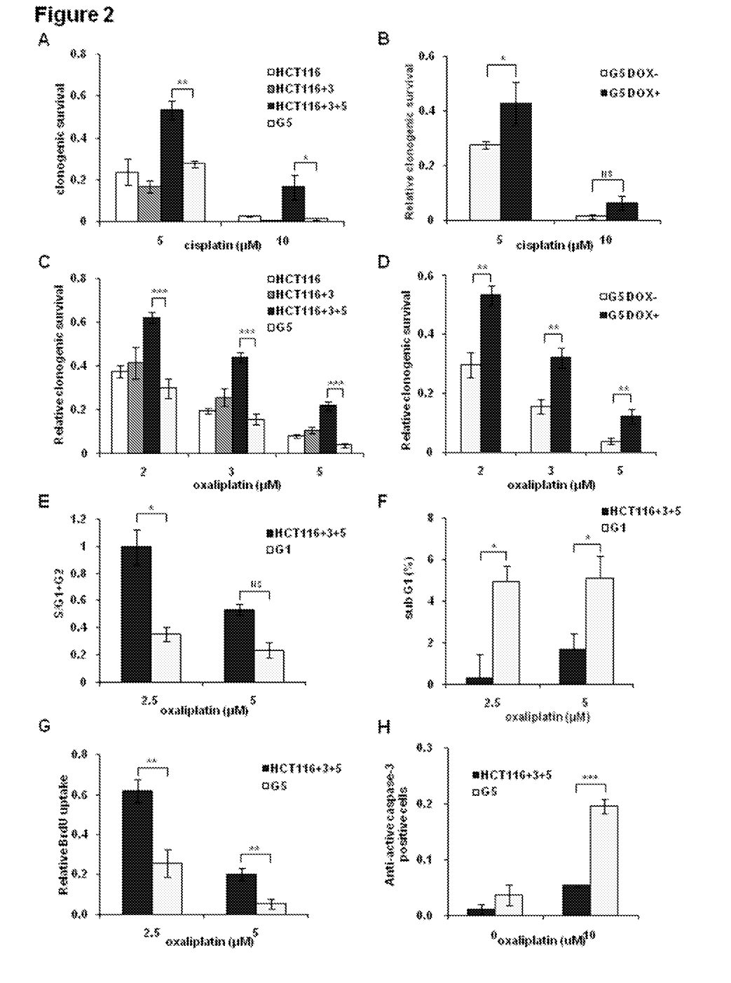

MSH3-deficient cells are more sensitive to cisplatin and oxaliplatin than MSH3-proficient cells. To determine whether MSH3 status affects cellular sensitivity to two platinum drugs, the clonogenic survival of HCT116 and HCT116-derived cell lines in cisplatin treated cells was examined. No significant differences in cisplatin sensitivity were observed between MLH1 and MSH3-deficient HCT116 and MLH1-only deficient HCT116+3 cell lines, whereas higher resistance was observed in MSH3-proficient HCT116+3+5 cell lines (FIG. 2A). Among various cell lines, the MSH3-deficient G5 clone was more sensitive than its parental HCT116+3+5 (FIG. 2A). To further confirm that MSH3 existence influenced cytotoxicity-induced by cisplatin, the clonogenic survival of G5 cells in the presence and absence of doxycycline was compared. It was found that restoration of MSH3 expression desensitized the cells to cisplatin (5 μM; FIG. 2B). These results indicate that MSH3 depletion led to the sensitization of cells to cisplatin. Also analyzed was the clonogenic survival of the other clones, G1 and G2 and it was found that these clones behaved similarly to G5 (data not shown). This further strengthened the possible role of MSH3 in the cytotoxicity caused by cisplatin. Next, it was determined whether MSH3 deficiency also influenced cellular sensitivity to oxaliplatin. Surprisingly, the MSH3-deficient HCT116, HCT116+3 and G5 clones were significantly more sensitive to oxaliplatin than the parental HCT116+3+5, as was the case for cisplatin (FIG. 2C). Furthermore, it was observed that the restoration of MSH3 in the MSH3-deficient cells led to restoration of oxaliplatin insensitivity (FIG. 2D). Next, the rate of growth inhibition and the levels of apoptosis between MSH3-proficient and MSH3-deficient cells after oxaliplatin treatment were compared. The present inventors found that the degree of cell growth inhibition (FIG. 2E) and the levels of apoptosis were significantly higher (FIG. 2F) in MSH3-deficient cells than in MSH3-proficient cells, using flow cytometry. It also confirmed cell proliferation was decreased and apoptotic fraction was increased in MSH3-deficient cells treated with oxaliplatin, using a BrdU assay and an immunofluorescense, respectively. These results are consistent with the findings shown herein on growth inhibition results obtained via clonogenic assays.

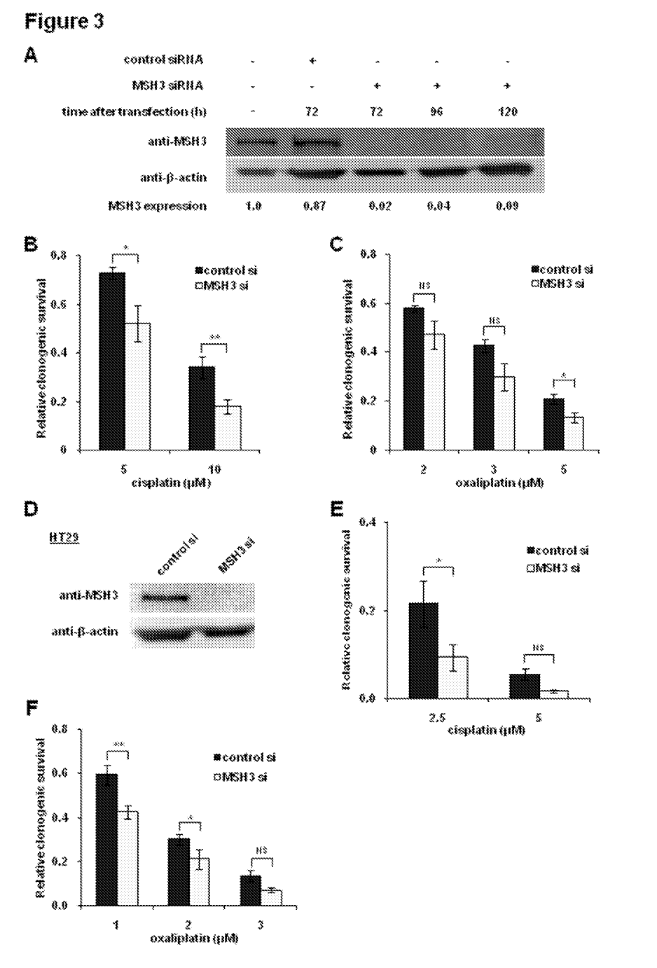

Depletion of MSH3 by siRNA transfection also sensitizes cells to cisplatin and oxaliplatin. To further confirm the role of MSH3-related sensitization to cisplatin and oxaliplatin, the clonogenic survival frequencies of cells transiently transfected with MSH3 siRNA and non-targeted siRNA were determined. In these studies, it is shown that MSH3 protein expression was significantly diminished 72 hours after siRNA transfection (98% MSH3 expression inhibition compared to untreated cells) in HCT116+3+5 cells (FIG. 3A). HCT116+3+5 cells were transfected with MSH3 siRNA and the cells were exposed to cisplatin (5 μM and 10 μM) or oxaliplatin (2 μM and 5 μM) 48 hours after transfection. As shown in FIGS. 3B & 3C, transfection of MSH3 siRNA rendered HCT116+3+5 cells more susceptible to both cisplatin and oxaliplatin in comparison to cell lines transfected with non-targeted siRNA. To further confirm this increased sensitivity to platinum drugs in MSH3-depleted cells, another colon cancer cell line, HT29, was transfected with MSH3 siRNA or non-targeted siRNA. It was confirmed that MSH3 was repressed almost completely in HT29 cells (FIG. 3D) and that HT29 cells treated with MSH3 siRNA became more sensitive to both cisplatin and oxaliplatin (FIGS. 3E & 3F). These results further strengthen the findings that MSH3 deficiency sensitizes cells to both cisplatin and oxaliplatin.

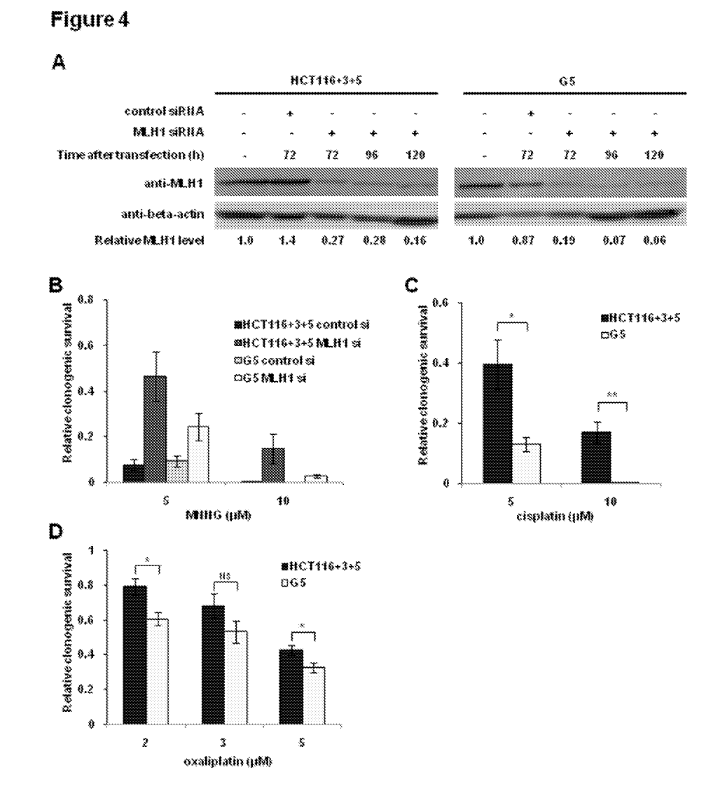

Cisplatin or oxaliplatin sensitivity in MSH3-proficient and MSH3-deficient cells occurs independently of MLH1 status in colon cancer cells. From a clinical point of view, it is important to determine whether the MSH3 status influences sensitivity to cisplatin and oxaliplatin in patients with cancers that are also MLH1-deficient. To address this question, the sensitivity of MSH3-deficient G5 cells and MSH3-proficient cells to cisplatin and oxaliplatin by inducing siRNA mediated down-regulation of MLH1 expression were compared (FIG. 4A). When MLH1 was down-regulated in both HCT116+3+5 and G5 cells transfected with MLH1 siRNA, both cell lines became more resistant to MNNG in comparison to untreated control cell lines (FIG. 4B), validating the functional repression of MLH1 in these conditions (10,11). Interestingly, in this scenario, it was observed that G5 cells were more sensitive to cisplatin and oxaliplatin (2 μM and 5 μM) than HCT116+3+5 (FIGS. 4C & 4D). These results demonstrate that MSH3-dependent sensitivity to cisplatin and oxaliplatin occurs independently of MLH1 status.

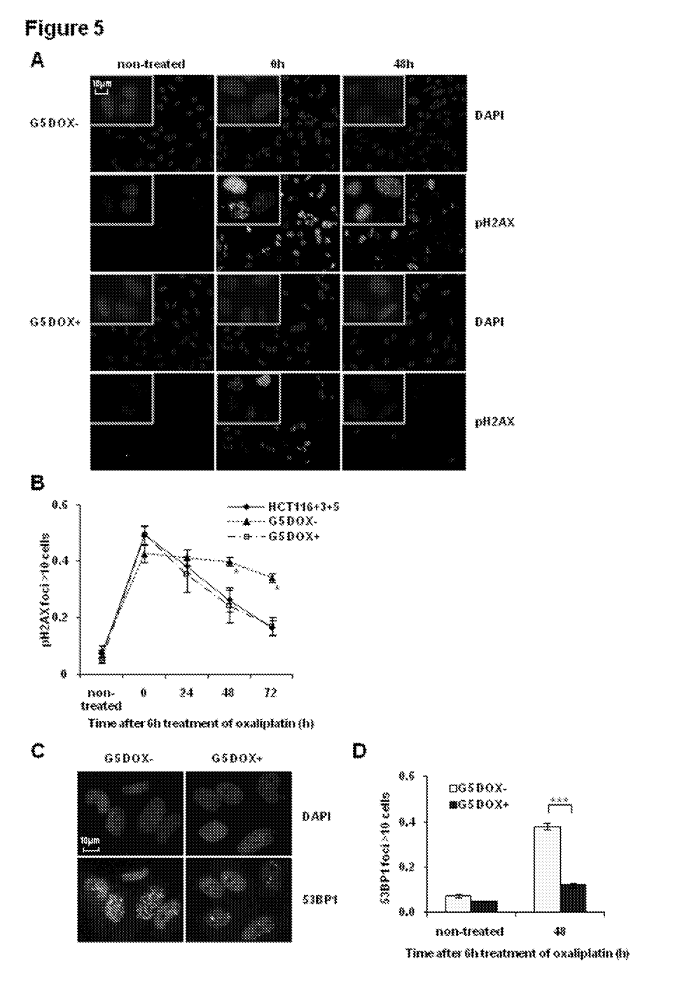

MSH3-deficient cells demonstrate sustained levels of pH2AX and 53BP1 after oxaliplatin treatment. Platinum drugs induce DNA intrastrand crosslinks and ICLs, and some of the lesions eventually lead to secondary DNA double stranded breaks (DSBs), presumably as a result of a collapsed replication folk (24). To determine whether MSH3 is involved in the repair of DSBs, the time-dependent changes in the levels of nuclear pH2AX, a surrogate marker for DNA DSBs (25), using immunofluorescence staining was analyzed. It was found that there were no differences in the number of pH2AX foci-positive cells before and after oxaliplatin treatment in the MSH3-proficient and -deficient cell lines. In contrast, it was observed that a lower rate of reduction in the number of pH2AX foci-positive cells in the MSH3-deficient G5 cells compared to both MSH3-restored G5 cells and the HCT116+3+5 cell lines during 48 and 72 hours treatment with oxaliplatin (FIGS. 5A & 5B), indicating that DSB repair is compromised only in MSH3-deficient cell lines. To further confirm this DSB repair inefficiency, immunofluorescence assays were performed using an anti-53BP1 antibody, another marker for detecting DNA DSB. Sustained levels of 53BP1 in MSH3-deficient G5 cells after oxaliplatin treatment were confirmed (FIGS. 5C & 5D). These results show that the higher sensitivity of MSH3-deficient cells to oxaliplatin may in part be due to a reduced DNA DSB repair efficiency, rather than a quantitative difference in the burden of DNA damage induced by treatment.

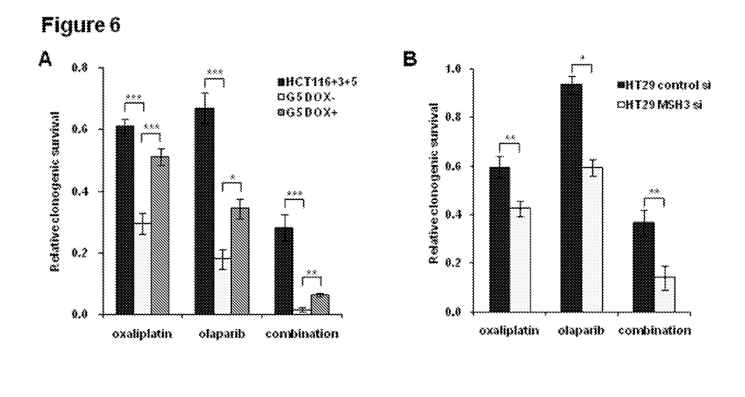

MSH3-deficient cells are also sensitive to olaparib, a PARP inhibitor. PARP inhibitors increase the number of single strand breaks, which eventually leads to DNA DSBs that are repaired by the HR system. HR-defective cells are hypersensitive to PARP inhibitors because of their inability to repair these DSBs (18,19). The possible role of MSH3 in DSB repair evidenced from the results (FIG. 5) prompted further examination of whether MSH3-deficient cells are also sensitive to PARP inhibitors. As shown in FIG. 6A, MSH3-deficient G5 cells were more sensitive to olaparib than the MSH3-restored G5 cell line. These data clearly support the role of MSH3 in DSB repairs in CRC cells. Moreover, the combination of oxaliplatin and olaparib exhibited a synergistic effect in cytotoxicity in the MSH3-deficient G5 cells compared to the parental HCT116+3+5 cells. This effect was confirmed with two other colon cancer cell lines HT29 (FIG. 6B) and SW480 (data not shown) in a transient knockdown system using MSH3 siRNA. These results demonstrate a combination therapy of platinum drugs and PARP inhibitors in MSH3-deficient cancers.

The present study elucidated that a loss of MSH3 affects cellular sensitivity caused by platinum drugs. This observation can be used to establish diagnostic and therapeutic strategies that MSH3 status may be used as a predictive marker for chemotherapeutic outcome in patients with MSH3-deficient tumors. Briefly, using the isogenic colon cancer cell lines in which MSH3 expression is regulated by a tet-off system, it was demonstrated that the depletion of MSH3 expression in colon cancer cells sensitized them to not only cisplatin, but also to oxaliplatin and a PARP inhibitor. These data suggest that these effects can be best explained by the reduced ability of MSH3-deficient cells to repair DSBs that are incurred following treatment with these drugs, although the precise mechanisms by which MSH3 is involved for DNA DSB repair require further exploration. This is the first demonstration that selective inhibition of MSH3 increases cellular sensitivity to platinum drugs and PARP inhibitor. Moreover, these results demonstrate that the MSH3-dependent increase in sensitivity to cisplatin and oxaliplatin is not influenced by down-regulation of MLH1 and is probably independent of the canonical MMR system.

The role of MutS and MutL homologues in repair for ICLs has been well-studied using psoralen ICLs (12-15,26). These data suggest that MutSβ is involved in both recognition and processing of certain types of ICLs in cooperation with other proteins such as NER and HR proteins, and the fact that MutSβ also functions in ICL repair independent of its primary role in MMR. The findings herein show that MSH3-depleted cells are sensitive to cisplatin and oxaliplatin, and this occurs independent of MLH1 function is consistent with these findings using psoralen ICLs.

These results show that MutSβ is involved in repair of toxic DSBs induced by ICL adducts. First, there is existing evidence that MSH3 gets co-localized to DSB lesions induced by laser (27) and by carcinogens such as chromium(VI) (28). Second, the present inventors recognized that sustained levels of pH2AX and 53BP1 that co-localize with DSBs in MSH3-deficient cells after oxaliplatin treatment compared with MSH3-proficient cells. Third, MSH3-deficient cells are sensitive to a PARP inhibitor which induces DSBs. Thus, these results show that unrepaired DSBs due to MSH3 deficiency are the direct cause of cell death. However, a recent study has shown that tumors occurring in MSH2-null mice are more resistant to cisplatin and the combination of 5-FU plus oxaliplatin than tumors in mice that have MSH2 G674D mutations (29). Interestingly, although this missense mutation results in loss of MMR activity, it still retains sensitivity to the DNA damage. These results show that MSH2 has distinctive functions in MMR activity and chemosensitivity (29). MSH2 and MLH1 have been shown to be required for the activation of various proteins involved in apoptotic pathways such as JNK and c-Abl after cisplatin treatment (30), however, it is not clear whether MutSα or MutSβ or both are involved in the signaling pathways caused by cisplatin or oxaliplatin. However, since the results indicate that loss of MSH3 increases the sensitivity to cisplatin and oxaliplatin, it is likely that MutSβ is mainly involved in the repair of DNA damage and MutSα is involved in both the repair and signaling pathways that lead to cell death. Further studies may elucidate the exact role of MutSα or MutSβ in repair for DNA damage and in damage signaling caused by these drugs.

The results regarding the sensitivity of MSH3-deficient cells to cisplatin and oxaliplatin are inconsistent with a previous report by Vaisman et al. (31). That study reported that the sensitivity to these drugs did not differ between the MSH3-deficient HHUA cells and the MSH3-proficient HHUA complemented with chromosome 5 (31). In their study, the influence by hundreds of other genes of chromosome 5 could not be excluded; therefore the data contained herein are more robust as isogenic clones of HCT116 colon cancer cells were used, in which MSH3 expression was selectively regulated as needed.

From a clinical standpoint, the results shown herein demonstrate that a considerable population of patients with MSI CRC might benefit from oxaliplatin-based treatment regimens, PARP inhibitors, or in particular, a combination of the two. In CRC, many recent studies have shown that patients with stage III MSI cancer do not benefit from 5-FU adjuvant chemotherapy (32-34). Moreover, Bertagnolli et al., reported that patients with stage III MSI-CRC benefit from adjuvant chemotherapy containing 5-FU and irinotecan (35) whereas another study has reported that these patients received no benefit from this adjuvant treatment (36). These inconsistent results raise the possibility that there may be subgroups of patients that have different chemosensitivities among MSI CRC. For instance, these results show that there are at least two subpopulations of MLH1-deficient CRC, MSH3-proficient and MSH3-deficient CRC, and these may respond differentially to oxaliplatin, a PARP inhibitor and their combination depending on the MSH3 status.

In addition to MSH3, several other DNA repair genes are mutated in MSI cancers. MRE11A and RAD50, whose products are formed in the DSB repair complex MRE11A-hRAD50-NBS1, are among the most frequently mutated genes in MSI cancers (22). Mutations in MRE11A and RAD50 have been shown to increase sensitivity to irinotecan, which induces secondary DSBs, in cultured cells (37,38). These results show that MSH3 deficiency sensitized cells to SN-38, an active metabolite of irinotecan (data not shown). Moreover, loss of phosphatase and tensin homologue, another gene frequently mutated in MSI cancer, has been shown to sensitize cells to PARP inhibitors through inefficiency of HR repair (39,40). Thus, analyzing these genes or proteins that are involved in DSB repair could be helpful for predicting the therapeutic outcome in patients with MSI cancer. Clinical studies to validate predictive markers for drug therapy in MSI cancer are warranted.

Previously, the present inventors demonstrated that loss of MSH3 expression caused the EMAST and MSI-low phenotypes, and that focal loss of MSH3 expression was associated with EMAST in the sporadic CRC tissues (23). Moreover, most MSI-low CRCs and some proportion of MSS tumors exhibited EMAST, suggesting that these tissues might have experienced MSH3 deficiency (23). MSH3 deficiency is possibly related to disease progression in MLH1-deficient CRC (20), and MSI-low CRC have poor prognosis (41,42), raising the possibility that loss of MSH3 may be related to promotion of metastasis or recurrence of CRC. In this scenario, treatment of sporadic CRC containing MSH3-negative cancer cell populations with platinum drugs or PARP inhibitors, or both, may inhibit disease progression.

In conclusion, it is demonstrated herein that MSH3-deficient cells are sensitive to cisplatin, oxaliplatin and a PARP inhibitor possibly resulting from reduced repair for DNA DSBs. These findings contribute to a better understanding of the role of MSH3 for DNA repair and drug sensitivity, and to predicting and improving the therapeutic outcome of patients with MSH3-deficient cancers.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,252,955: Method of Detecting Colon Cancer.
U.S. Pat. No. 7,575,928: Genes for Diagnosing Colorectal Cancer.
U.S. Pat. No. 7,022,472: Mutations in Human MLH1 and Human MSH2 Genes useful in Diagnosing Colorectal Cancer.
U.S. Patent Application No. 20090305277: Gene Expression Markers For Prediction Of Patient Response To Chemotherapy.
1. Kunkel, T. A., and Erie, D. A. (2005) Annu Rev. Biochem. 74, 681-710
2. Jiricny, J. (2006) Nat. Rev. Mol. Cell. Biol. 7, 335-346
3. Hendriks, Y. M., de Jong, A. E., Morreau, H., Tops, C. M., Vasen, H. F., Wijnen, J. T., Breuning, M. H., and Brocker-Vriends, A. H. (2006) CA Cancer J. Clin. 56, 213-225
4. Boland, C. R., and Goel, A. (2010) Gastroenterology 138, 2073-2087 e2073
5. Black, D., Soslow, R. A., Levine, D. A., Tomos, C., Chen, S. C., Hummer, A. J., Bogomolniy, F., Olvera, N., Barakat, R. R., and Boyd, J. (2006) J. Clin. Oncol. 24, 1745-1753
6. Pal, T., Permuth-Wey, J., Kumar, A., and Sellers, T. A. (2008) Clin. Cancer Res. 14, 6847-6854
7. Duckett, D. R., Drummond, J. T., Murchie, A. I., Reardon, J. T., Sancar, A., Lilley, D. M., and Modrich, P. (1996) Proc. Natl. Acad. Sci. U.S.A. 93, 6443-6447
8. de Wind, N., Dekker, M., Claij, N., Jansen, L., van Klink, Y., Radman, M., Riggins, G., van der Valk, M., van't Wout, K., and to Riele, H. (1999) Nat. Genet. 23, 359-362
9. Abuin, A., Zhang, H., and Bradley, A. (2000) Mol. Cell. Biol. 20, 149-157
10. Koi, M., Umar, A., Chauhan, D. P., Cheman, S. P., Carethers, J. M., Kunkel, T. A., and Boland, C. R. (1994) Cancer Res. 54, 4308-4312
11. Hawn, M. T., Umar, A., Carethers, J. M., Marra, G., Kunkel, T. A., Boland, C. R., and Koi, M. (1995) Cancer Res. 55, 3721-3725
12. Zhang, N., Lu, X., Zhang, X., Peterson, C. A., and Legerski, R. J. (2002) Mol. Cell. Biol. 22, 2388-2397
13. Zhao, J., Jain, A., Iyer, R. R., Modrich, P. L., and Vasquez, K. M. (2009) Nucleic Acids Res. 37, 4420-4429
14. Vasquez, K. M. (2010) Environ. Mol. Mutagen. 51, 527-539
15. Zhang, N., Liu, X., Li, L., and Legerski, R. (2007) DNA Repair (Amst) 6, 1670-1678
16. Zhu, G., and Lippard, S. J. (2009) Biochemistry 48, 4916-4925
17. Chaney, S. G., Campbell, S. L., Bassett, E., and Wu, Y. (2005) Crit. Rev. Oncol. Hematol. 53, 3-11
18. Bryant, H. E., Schultz, N., Thomas, H. D., Parker, K. M., Flower, D., Lopez, E., Kyle, S., Meuth, M., Curtin, N.J., and Helleday, T. (2005) Nature 434, 913-917
19. Farmer, H., McCabe, N., Lord, C. J., Tutt, A. N., Johnson, D. A., Richardson, T. B., Santarosa, M., Dillon, K. J., Hickson, I., Knights, C., Martin, N. M., Jackson, S. P., Smith, G. C., and Ashworth, A. (2005) Nature 434, 917-921
20. Plaschke, J., Kruger, S., Jeske, B., Theissig, F., Kreuz, F. R., Pistorius, S., Saeger, H. D., Iaccarino, I., Marra, G., and Schackert, H. K. (2004) Cancer Res. 64, 864-870
21. Miguel, C., Jacob, S., Grandjouan, S., Aime, A., Viguier, J., Sabourin, J. C., Sarasin, A., Duval, A., and Praz, F. (2007) Oncogene 26, 5919-5926
22. Hewish, M., Lord, C. J., Martin, S. A., Cunningham, D., and Ashworth, A. (2010) Nat. Rev. Clin. Oncol. 7, 197-208
23. Haugen, A. C., Goel, A., Yamada, K., Marra, G., Nguyen, T. P., Nagasaka, T., Kanazawa, S., Koike, J., Kikuchi, Y., Zhong, X., Arita, M., Shibuya, K., Oshimura, M., Hemmi, H., Boland, C. R., and Koi, M. (2008) Cancer Res. 68, 8465-8472
24. Hinz, J. M. (2010) Environ. Mol. Mutagen. 51, 582-603
25. Bonner, W. M., Redon, C. E., Dickey, J. S., Nakamura, A. J., Sedelnikova, O. A., Solier, S., and Pommier, Y. (2008) Nat. Rev. Cancer 8, 957-967
26. Wu, Q., and Vasquez, K. M. (2008) PLoS Genet. 4, e1000189
27. Hong, Z., Jiang, J., Hashiguchi, K., Hoshi, M., Lan, L., and Yasui, A. (2008) J. Cell Sci. 121, 3146-3154
28. Reynolds, M. F., Peterson-Roth, E. C., Bespalov, I. A., Johnston, T., Gurel, V. M., Menard, H. L., and Zhitkovich, A. (2009) Cancer Res. 69, 1071-1079
29. Kucherlapati, M. H., Lee, K., Nguyen, A. A., Clark, A. B., Hou, H., Jr., Rosulek, A., Li, H., Yang, K., Fan, K., Lipkin, M., Bronson, R. T., Jelicks, L., Kunkel, T. A., Kucherlapati, R., and Edelmann, W. (2010) Gastroenterology 138, 993-1002 e1001
30. Nehme, A., Baskaran, R., Nebel, S., Fink, D., Howell, S. B., Wang, J. Y., and Christen, R. D. (1999) Br. J. Cancer 79, 1104-1110
31. Vaisman, A., Varchenko, M., Umar, A., Kunkel, T. A., Risinger, J. I., Barrett, J. C., Hamilton, T. C., and Chaney, S. G. (1998) Cancer Res. 58, 3579-3585
32. Ribic, C. M., Sargent, D. J., Moore, M. J., Thibodeau, S, N., French, A. J., Goldberg, R. M., Hamilton, S. R., Laurent-Puig, P., Gryfe, R., Shepherd, L. E., Tu, D., Redston, M., and Gallinger, S. (2003) N. Engl. J. Med. 349, 247-257
33. Popat, S., Hubner, R., and Houlston, R. S. (2005) J. Clin. Oncol. 23, 609-618
34. Sargent, D. J., Marsoni, S., Monges, G., Thibodeau, S, N., Labianca, R., Hamilton, S. R., French, A. J., Kabat, B., Foster, N. R., Torri, V., Ribic, C., Grothey, A., Moore, M., Zaniboni, A., Seitz, J. F., Sinicrope, F., and Gallinger, S. (2010) J. Clin. Oncol. 28, 3219-3226
35. Bertagnolli, M. M., Niedzwiecki, D., Compton, C. C., Hahn, H. P., Hall, M., Damas, B., Jewell, S. D., Mayer, R. J., Goldberg, R. M., Saltz, L. B., Warren, R. S., and Redston, M. (2009) J. Clin. Oncol. 27, 1814-1821
36. Tejpar, S., Bosman, F., Delorenzi, M., Fiocca, R., Yan, P., Klingbiel, D., Dietrich, D., Van Cutsem, E., Labianca, R., and Roth, A. (2009) J. Clin. Oncol. 27, 4001
37. Vilar, E., Scaltriti, M., Balmana, J., Saura, C., Guzman, M., Arribas, J., Baselga, J., and Tabernero, J. (2008) Br. J. Cancer 99, 1607-1612
38. Rodriguez, R., Hansen, L. T., Phear, G., Scorah, J., Spang-Thomsen, M., Cox, A., Helleday, T., and Meuth, M. (2008) Clin. Cancer Res. 14, 5476-5483
39. Mendes-Pereira, A. M., Martin, S. A., Brough, R., McCarthy, A., Taylor, J. R., Kim, J. S., Waldman, T., Lord, C. J., and Ashworth, A. (2009) EMBO Mol. Med. 1, 315-322
40. McEllin, B., Camacho, C. V., Mukherjee, B., Hahm, B., Tomimatsu, N., Bachoo, R. M., and Burma, S. (2010) Cancer Res. 70, 5457-5464
41. Kohonen-Corish, M. R., Daniel, J. J., Chan, C., Lin, B. P., Kwun, S. Y., Dent, O. F., Dhillon, V. S., Trent, R. J., Chapuis, P. H., and Bokey, E. L. (2005) J. Clin. Oncol. 23, 2318-2324
42. Wright, C. M., Dent, O. F., Newland, R. C., Barker, M., Chapuis, P. H., Bokey, E. L., Young, J. P., Leggett, B. A., Jass, J. R., and Macdonald, G. A. (2005) Gut 54, 103-108

What is claimed is:

1. A method for treating a patient at risk for or diagnosed with one or more adenocarcinomas, the method comprising:
determining the overall expression of MSH3 in cells suspected of being adenocarcinoma cells obtained from the patient; and
predicting the efficacy of therapy with an anti-neoplastic agent for treating the patient, wherein a decrease in the overall expression of MSH3 in the patient cells when compared to the expression of MSH3 in normal cells indicates a predisposition to responsiveness to anti-neoplastic agent therapy, wherein the therapy comprises administering an effective amount of the anti-neoplastic agent to the patient.

2. The method of claim 1, wherein the adenocarcinomas are selected from the group consisting of colorectal cancer (CRC), lung cancer, cervical cancer, ovarian cancer, prostate cancer, kidney cancer, liver cancer, testicular cancer, bladder cancer, vaginal cancer, breast cancer, esophageal cancer, pancreatic cancer, stomach cancer or a solid tumor.

3. The method of claim 1, wherein the step of determining the overall level of expression of MSH3 using at least one of determining MSH3 protein expression, MSH3 nucleic acid expression, performing mass spectrometry analysis of MSH3 nucleic acids obtained from the individual, performing rolling circle amplification of a portion of a MSH3 nucleic acid obtained from the individual, performing hybridization with an allele specific probe, performing hybridization with an antibody probe, or performing immunohistochemistry.

4. The method of claim 1, wherein the anti-neoplastic agent is selected from at least one of 1,3-bis(2-chloroethyl)-1-nitrosourea, busulfan, carmustine, chlorambucil, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, temozolomide, topotecan, and ionizing radiation, interstrand crosslinking agents, cisplatin, carboplatin, oxaliplatin, furocoumarins, psoralen, poly (ADP-ribose) polymerase (PARP) inhibitors, olaparib, isoindolinone derivatives, veliparib, iniparib, or 4-methoxy-carbazole derivatives.

5. A method for selecting a cancer therapy for a patient at risk for or diagnosed with colorectal cancer, the method comprising:
determining the overall expression level of MSH3 of the patient with colorectal cancer and predicting the efficacy of therapy with an anti-neoplastic agent for treating the patient with an anti-neoplastic agent, wherein a decrease in the overall level of expression of MSH3 indicates that the DNA crosslinking agent is a suitable therapy for the colorectal cancer of the patient.

6. The method of claim 5, wherein the step of determining the overall level of expression of MSH3 using at least one of determining MSH3 protein expression, MSH3 nucleic acid expression, performing mass spectrometry analysis of MSH3 nucleic acids obtained from the individual, performing rolling circle amplification of a portion of a MSH3 nucleic acid obtained from the individual, performing hybridization with an allele specific probe, performing hybridization with an antibody probe, or performing immunohistochemistry.

7. The method of claim 5, wherein the anti-neoplastic agent is selected from at least one of 1,3-bis(2-chloroethyl)-1-nitrosourea, busulfan, carmustine, chlorambucil, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, temozolomide, topotecan, and ionizing radiation, interstrand crosslinking agents, cisplatin, carboplatin, oxaliplatin, furocoumarins, psoralen, poly (ADP-ribose) polymerase (PARP) inhibitors, olaparib, isoindolinone derivatives, veliparib, iniparib, or 4-methoxy-carbazole derivatives.

8. A method for stratifying a patient in a subgroup of a clinical trial of a cancer therapy, the method comprising:
determining the overall expression of MSH3 in cells suspected of being cancer cells from the patient; and
predicting the efficacy of therapy with a candidate drug for treating the patient, wherein a decrease in the overall expression of MSH3 in the patient cells when compared to the expression of MSH3 in normal cells indicates a predisposition to responsiveness to therapy with the candidate drug, wherein the therapy comprises administering an effective amount of the candidate drug to patients and the level of expression of MSH3 enables the stratification of the patient into a subgroup for the clinical trial.

9. The method of claim 8, wherein the cancer cells are selected from at least one of colorectal cancer (CRC), lung cancer, cervical cancer, ovarian cancer, prostate cancer, kidney cancer, liver cancer, testicular cancer, bladder cancer, vaginal cancer, breast cancer, esophageal cancer, pancreatic cancer, stomach cancer or a solid tumor.

10. The method of claim 8, wherein the step of determining the overall level of expression of MSH3 using at least one of determining MSH3 protein expression, MSH3 nucleic acid expression, performing mass spectrometry analysis of MSH3 nucleic acids obtained from the individual, performing rolling circle amplification of a portion of a MSH3 nucleic acid obtained from the individual, performing hybridization with an allele specific probe, performing hybridization with an antibody probe, or performing immunohistochemistry.

11. The method of claim 8, wherein the candidate agent is a genotoxic agent.

12. The method of claim 8, wherein the candidate agent is a poly (ADP-ribose) polymerase (PARP) inhibitor.

13. A method for stratifying a patient in a subgroup of colorectal cancer, the method comprising:
determining the overall expression of MSH3 in cells suspected of being colorectal cancer cells from the patient; and
predicting the stage of the colorectal, wherein a decrease in the overall expression of MSH3 in the patient cells when compared to the expression of MSH3 in normal colorectal cells disease progression

14. The method of claim 13, wherein disease progression and a decrease in MSH3 expression indicates a predisposition of the colorectal cancer to an anti-neoplastic agent therapy.

15. A method for treating a patient at risk for or diagnosed with colorectal cancer, the method comprising:
determining the overall expression of MSH3 in cells suspected of being colorectal cancer cells from the patient which indicates a predisposition to responsiveness to therapy with one or more DNA crosslinking agents;
determining a continued decrease in the overall expression of MSH3 in the patient; and
administering a therapeutically effective amount of a DNA crosslinking agent in an amount sufficient to eliminate colorectal cancer cells with decreases MSH3 expression.

16. A method of performing a clinical trial to evaluate a candidate drug believed to be useful in treating a disease state associated with MSH3 gene expression, the method comprising:
a) measuring the level of MSH3 expression from tissue suspected of having colorectal cancer from a set of patients;
b) administering a candidate drug to a first subset of the patients, and a placebo to a second subset of the patients;
c) repeating step a) after the administration of the candidate drug or the placebo; and
d) determining if the candidate drug reduces the number of colorectal cells that have a decrease in the expression of MSH3 that is statistically significant as compared to any reduction occurring in the second subset of patients, wherein a statistically significant reduction indicates that the candidate drug is useful in treating said disease state.

17. A method for determining whether if a colorectal cancer is likely to be resistant or responsive to a DNA damaging agent for the treatment of colorectal cancer, the method comprising the step(s) of:
obtaining a biological sample from the a patient suspected of having colorectal cancer;
examining a biological sample from the cancer for a decrease in the overall expression of MSH3; and
identifying the colorectal cancer as having an enhanced susceptibility to the DNA damaging agent where there is decreased expression or activity of MSH3 relative to the same biomarker's expression or activity level in the colorectal cancer that is responsive to the DNA damaging agent.

18. A biomarker for colorectal cancer disease progression, wherein the biomarker is MSH3 and a decrease in the overall expression of MSH3 in colorectal cancer cells obtained from a patient is indicative of colorectal cancer disease progression when compared to MSH3 expression is normal colorectal cancer cells or colorectal cancer cells obtained at an earlier timepoint from the same patient.

19. The biomarker of claim 18, wherein the step of determining the overall level of expression of MSH3 using at least one of determining MSH3 protein expression, MSH3 nucleic acid expression, performing mass spectrometry analysis of MSH3 nucleic acids obtained from the individual, performing rolling circle amplification of a portion of a MSH3 nucleic acid obtained from the individual, performing hybridization with an allele specific probe, performing hybridization with an antibody probe, or performing immunohistochemistry.

20. A kit for a diagnosis of colorectal cancer comprising biomarker detecting reagents for determining a differential expression level of MSH3 and instructions for their use in diagnosing risk for colorectal cancer.

21. The kit of claim 20, wherein both MSH3 mRNA and protein expression levels in a sample from a patient at risk for colorectal is significantly decreased compared to that of a normal subject.

22. The kit of claim 20, wherein the MSH3 mRNA expression level is decreased in the patient at risk for colorectal cancer in comparison a normal subject.

23. The kit of claim 20, wherein the MSH3 protein expression level is decreased in the patient as at risk for colorectal cancer in comparison to a normal subject.

24. A method for diagnosing or detecting colorectal cancer progression in a human subject comprising the steps of:
identifying the human subject suspected of suffering from colorectal cancer;
obtaining one or more biological samples from the subject, wherein the biological samples are selected from the group consisting of a tissue sample, a fecal sample, a cell homogenate, and one or more biological fluids comprising;
measuring an overall expression pattern of MSH3 in one or more cells obtained from the biological samples of the subject; and
comparing the overall expression pattern of the MSH3 from the biological sample of the subject suspected of suffering from colorectal cancer with the overall expression pattern of MSH3 from a biological sample of a normal subject, wherein the normal subject is a healthy subject not suffering from colorectal cancer, wherein a decrease in the overall expression pattern of the MSH3 in the biological sample of the subject is indicative of the presence, risk for developing or both of colorectal cancer.

25. The method of claim 24, wherein a significant decrease in the expression levels of MSH3 mRNA, MSH3 protein or both, are indicative of the presence, risk for developing or both of invasive colorectal cancer.

26. The method of claim 24, wherein the step of determining the overall level of expression of MSH3 comprises analyzing the one or more cells from the biological sample for MSH3 nucleic acid expression.

27. The method of claim 24, wherein the step of determining the overall level of expression of MSH3 comprises performing mass spectrometry analysis of MSH3 nucleic acids obtained from the subject.

28. The method of claim 24, wherein the step of determining the overall level of expression of MSH3 comprises performing a rolling circle amplification of a portion of a MSH3 nucleic acid obtained from the subject.

29. The method of claim 24, wherein the step of determining the overall level of expression of MSH3 comprises hybridization with an allele specific probe or an antibody probe.

30. The method of claim 24, wherein the step of determining the overall level of expression of MSH3 comprises immunohistochemistry.

31. The method of claim 24, wherein the method is used for treating a patient at risk or suffering from colorectal cancer, selecting a DNA crosslinking agent therapy for a patient at risk or suffering from colorectal cancer, stratifying a patient in a subgroup of colorectal cancer or for a colorectal cancer therapy clinical trial, determining resistivity or responsiveness to a colorectal cancer therapeutic regimen, developing a kit for diagnosis of colorectal cancer or any combinations thereof.
